(19) United States
(12) Patent Application Publication
(10) Pub. No.: US 2010/0135907 A1
(43) Pub. Date: Jun. 3, 2010

(54) Oral Drug Compliance Monitoring Using Sound Detection

(76) Inventors: Paul E. Cranley, Lake Jackson, TX (US); Larry S. Sun, Lake Jackson, TX (US); Malcolm W. Warren, Sandford, MI (US); Michelle A. Pressler, Lake Jackson, TX (US); Kristine L. Danowski, Midland, MI (US); Hamed Lakrout, Midland, MI (US); Christopher M. Jones, Midland, MI (US); Doug P. White, Midland, MI (US); Flor A. Castillo, Lake Jackson, TX (US); Bettina M. Rosner, Aerzen (DE)

Correspondence Address: The Dow Chemical Company, Intellectual Property Section, P.O. Box 1967, Midland, MI, 48641-1967, US

(21) Appl. No.: 11/989,225

(22) PCT Filed: Jul. 21, 2006
(86) PCT No.: PCT/US06/28513

§ 371 Date: Apr. 24, 2009

Related U.S. Application Data
(60) Provisional application No. 60/701,707, filed on Jul. 22, 2005.

(51) Int. Cl. A61K 49/00; A61P 43/00

(52) U.S. Cl. 424/9.1

(57) ABSTRACT

A tablet, pill or capsule containing a material which produces sound waves when the tablet, pill or capsule is exposed to the gastrointestinal system. A two step method for oral drug compliance monitoring. The first step is to ingest a tablet, pill or capsule containing a material which produces sound waves when the tablet, pill or capsule is exposed to the gastrointestinal system of a person. The second step is to detect the sound waves produced when the tablet, pill or capsule is exposed to the gastrointestinal system to confirm that the person has ingested the tablet, pill or capsule.

### CROSS-REFERENCE TO RELATED APPLICATION

This application claims the benefit of U.S. Provisional Application No. 60/701,707, filed Jul. 22, 2005.

### BACKGROUND OF THE INVENTION

The instant invention relates to oral drug compliance monitoring, and, more particularly, to a means for the detection of a material formulated into a drug tablet, pill or capsule that generates sound waves when the material is exposed to the environment of the gastrointestinal system.

Non-compliance of patients to drug regimens prescribed by their physicians results in increased cost of medical care, higher complication rates, as well as drug wastage. Non-compliance refers to the failure to take the prescribed dosage at the prescribed time which results in under medication or overmedication. In a survey of 57 non-compliance studies, non-compliance ranged from 15% to as high as 95% in all study populations, regardless of medications, patient population characteristics, drug being delivered or study methodology [Greenberg R N: Overview of patient compliance with medication dosing: A literature review. Clinical Therapeutics, 6(5):592-599, 1984].

In the clinical drug stage, accurately measuring compliance can lead to benefits such as: improved statistical reliability of a clinical study; clinical studies being completed sooner; and a determination of the effect of non-compliance as a function of the degree of non-compliance. In the therapeutic stage, accurately measuring compliance has a number of important benefits such as: warning a patient about the potential for developing a drug resistant infection related to poor compliance; and identifying a side effect of a drug related to overdosing.

Confirmation of drug compliance by way of direct observation by trained persons is effective but impractical in most situations. Confirmation of drug compliance by blood or urine analysis is also impractical in most situations. Transdermal detection devices attached to the skin of a patient have been developed which detect ingested drug components through the skin and such devices can transmit a signal to a remote receiver at an external site such as a healthcare facility, see U.S. Pat. No. 6,663,846 and USPAP 2005/0031536. Electronic sensor systems have been developed which detect ingested drug components in the breath of a patient, see USPAP 2004/0081587. Radio frequency identification (RFID) tags have been incorporated into drug pills, each tag capable of identifying the type of medication, its dosage, and its lot number by way of a unique code emitted by the tag when interrogated by a corresponding radio frequency “reader”, see U.S. Pat. No. 6,366,206.

Despite the many advances made in the prior art, it would be an advance in the art of drug compliance if a less complicated means could be discovered to determine drug compliance.

### SUMMARY OF THE INVENTION

The instant invention is a solution to the above stated problem. More specifically, the instant invention is an oral drug delivery system, comprising: a tablet, pill or capsule comprising sound generation means that produce sound waves when the tablet, pill or capsule is exposed to the gastrointestinal system. In another embodiment, the instant invention is a method for oral drug compliance monitoring, comprising the steps of: (a) ingesting a tablet, pill or capsule comprising a material which produces sound waves when the tablet, pill or capsule is exposed to the gastrointestinal system of a person; and (b) detecting the sound waves produced when the tablet, pill or capsule is exposed to the gastrointestinal system to confirm that the person has ingested the tablet, pill or capsule.

### BRIEF DESCRIPTION OF THE DRAWINGS

FIG. 1 is a cross-sectional side view of a pill or tablet containing granules of gasified candy;

FIG. 2 is a cross-sectional side view of a pill or tablet coated with a highly crystalline fractureable water permeable material;

FIG. 3 is a cross-sectional side view of a drug capsule containing granules of gasified candy;

FIG. 4 is a cross-sectional side view of a capsule containing a drug formulation, the capsule made from a highly crystalline fractureable water permeable material;

FIG. 5 is a schematic drawing of a sound sensor system;

FIG. 6 is a perspective view of a bag containing a sound sensor system adapted to be worn around the waist of a person;

FIG. 7 is a perspective view of a watch-like container containing a sound sensor system adapted to be worn around the wrist of a person; and

FIG. 8 is a perspective view of a pendent-like container containing a sound sensor system adapted to be worn around the neck of a person.

### DETAILED DESCRIPTION

The drug delivery system of the present invention comprises a tablet, pill or capsule comprising sound generation means that produce sound waves when the tablet, pill or capsule is exposed to the gastrointestinal system. Sound generation means include, for example, a material having properties that generate sound waves when exposed to water. Sound generation means also include a device capable of generating sound waves through electronic, hydraulic, or mechanical means. Examples of devices utilizing electronic means to generate sound waves include piezoelectric ultrasound generating devices commonly available, voice coil systems, speakers, and electric current systems. Examples of devices utilizing hydraulic means to generate sound waves include fluidic oscillators and similar devices such as a whistle. Examples of devices utilizing mechanical means to generate sound waves include hammer-like devices, tuning forks, and other devices utilizing a mechanism to hit a resonant object. Optimally, the sound generation means is capable of modulating the sound waves generated for the purposes of transmitting a serial number or a unique identifying signal associated with the specific pill, tablet, or capsule.

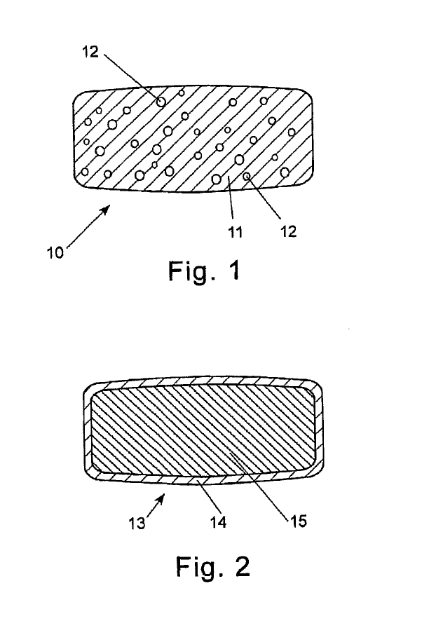

Referring now to FIG. 1, therein is shown a cross-sectional side view of a pill or tablet 10. The pill or tablet 10 comprises sound generation means such as, in the embodiment shown, a material that is granules of gasified candy 12. Optionally, the pill or tablet 10 comprises a drug formulation 11. When the pill or tablet 10 is ingested, it disperses in the gastrointestinal syslem and exposes the gasified candy 12 to water thereby releasing the gas trapped in the gasified candy to produce sound waves.

Gasified candy is commercially available under the trade name POP ROCKS. U.S. Pat. No. 4,289,794 (herein fully incorporated by reference) teaches a preferred method for preparing gasified candy.

Referring now to FIG. 2, therein is shown a cross-sectional side view of a pill or tablet 13. The pill or tablet 13 is coated with a highly crystalline fractureable water permeable material 14 and optionally comprises a drug formulation 15. When the pill or tablet 13 is ingested, water permeates into the pill or tablet 13 and eventually the highly crystalline fractureable water permeable material 14 fractures to produce sound waves.

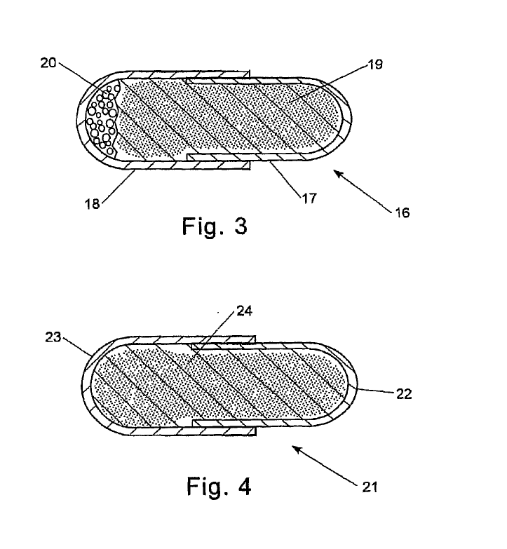

Highly crystalline fractureable water permeable material can be selected from appropriate grades of one or more of the following materials: ethyl cellulose, cellulose acetate and polylactidefglycolide copolymer. Referring now to FIG. 3, therein is shown a cross-sectional side view of a drug capsule 16. The drug capsule 16 contains granules of gasified candy 20 contained in gelatin capsule portions 17 and 18 and optionally contains a drug formulation 19. When the capsule 16 is ingested, it disperses in the gastrointestinal system and exposes the gasified candy 20 to water thereby releasing the gas trapped in the gasified candy to produce sound waves.

Referring now to FIG. 4, therein is shown a cross-sectional side view of a drug capsule 21. The drug capsule 21 comprises capsule portions 22 and 23. Capsule portions 22 and 23 are made of a highly crystalline fractureable water permeable material and optionally contain a drug formulation 24. When the capsule 21 is ingested, the capsule portions 22 and 23 are exposed to water. The water permeates into the capsule 21 eventually fracturing the highly crystalline fractureable water dispersible material to produce sound waves.

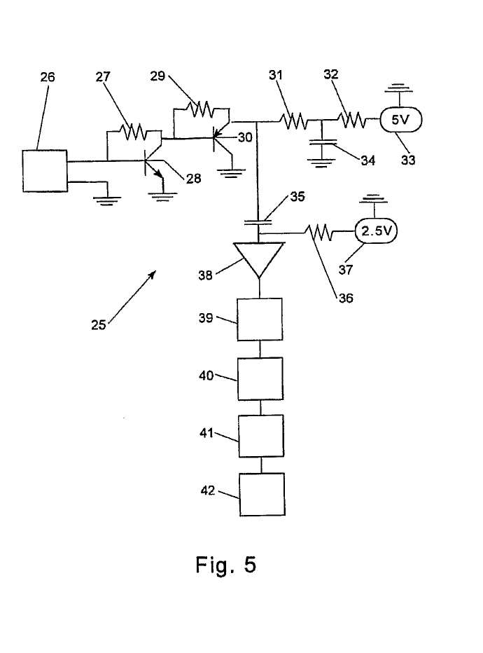

Referring now to FIG. 5, therein is shown a highly preferred sound sensor system 25 including a 9000 series piezo microphone 26 from Senscomp (Livonia, Mich.). One lead from the microphone 26 is grounded while the other lead is connected to a 10 M ohm resistor 27 and an MMBT5089 transistor 28. The resistor 27 and transistor 28 are connected to a 15 K ohm resistor 29 and a MMBT5087 transistor 30. A 5 volt direct current power source 33 is connected to a 10 K ohm resistor 32 which is connected to a 0.1 microfarad capacitor 34 and a 27 K ohm resistor 31. The resistor 29, the transistor 30 and the resistor 31 are connected to a 150 Pico Farad capacitor 35. A 2.5 volt direct current power source 37 is connected to the other lead of the capacitor 35 and to an operational amplifier 38 having a gain of 100. The output of the operational amplifier is passed through a 40 to 60 kilohertz band pass filter 39, through a level detector 40 and then to a microprocessor/data logger 41. The microprocessor/data logger 41 can be connected to (or communicate in a wireless manner) with a digital computer 42 for drug compliance monitoring at the patients residence and/or a health care facility.

The band pass filter 39 is highly preferred to filter out interfering sounds at lower frequencies that can come from the gastrointestinal system. The level detector 40 is highly preferred to filter out ultrasonic signals of a level too low to be caused by the fracturing of highly crystalline fractureable water permeable material or the sudden gas release of the gasified candy in the gastrointestinal system. Optimally, the sound sensor is capable of demodulating the sound waves and recovering a transmitted serial number or other unique identifying signal associated with the specific pill, tablet or capsule.

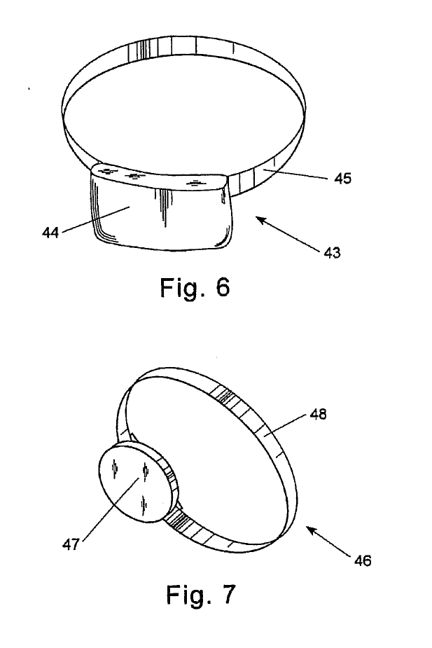

Referring now to FIG. 6, therein is shown a perspective view of a pack system 43 comprised of a belt 45 and a bag 44 containing the sound sensor system 25 of FIG. 5. The pack system 43 is adapted to be worn around the waist of a person. The pack system 43 is highly preferred because it places the microphone of the sound sensor system in relatively close proximity to the gastrointestinal system of the person wearing the pack system 43.

Referring now to FIG. 7, therein is shown a perspective view of a case system 46 comprised of a strap 48 and a case 47 containing the sound sensor system 25 of FIG. 5. The case system 46 is adapted to be worn around the wrist of a person. The case system 46 is convenient to wear but places the microphone of the sound sensor system relatively far from the gastrointestinal system of the person wearing the case system 46.

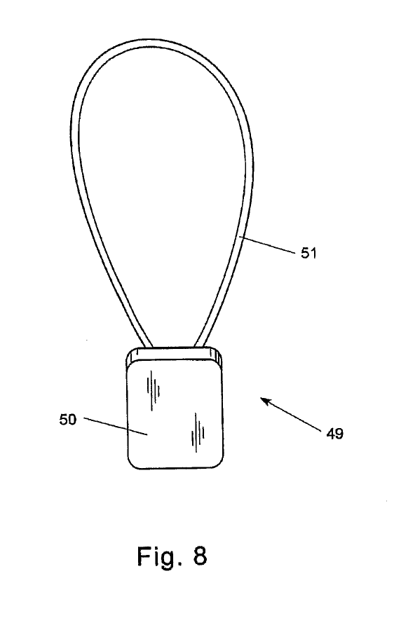

Referring now to FIG. 8, therein is shown a perspective view of a pendent system 49 comprised of a cord 51 and a pendent compartment 50 containing the sound sensor system 25 of FIG. 5. The pendent system 49 is adapted to be worn around the neck of a person. The pendent system 43 is more preferred than the case system 46 of FIG. 7 because it places the microphone of the sound sensor system in closer proximity to the gastrointestinal system of the person wearing the pendent system 49.

While the instant invention has been described above according to its preferred embodiments, it can be modified within the spirit and scope of this disclosure. For example, the case 47 of FIG. 7 could be adhesively attached to a convenient location on a patient's abdomen. This application is therefore intended to cover any variations, uses, or adaptations of the instant invention using the general principles disclosed herein. Further, the instant application is intended to cover such departures from the present disclosure as come within the known or customary practice in the art to which this invention pertains and which fall within the limits of the following claims.

What is claimed is:

1. An oral drug delivery system, comprising: a tablet, pill or capsule comprising sound generation means that produce sound waves when the tablet, pill or capsule is exposed to the gastrointestinal system.

2. The oral drug delivery system of claim 1, wherein the sound generation means is a material which produces sound waves when the tablet, pill or capsule is exposed to the gastrointestinal system.

3. The oral drug delivery system of claim 2, wherein the material is gasified candy.

4. The oral drug delivery system of claim 3, wherein the gasified candy is made by maintaining a sugar melt at a temperature below about 280° F. at a super atmospheric gas pressure effective to produce in the gasified candy observable gas bubbles wherein the majority of the observable gas bubbles have a diameter of above about 225 micrometers and wherein the gas is carbon dioxide, nitrogen or air.

5. The oral drug delivery system of claim 2, wherein the material which produces sound waves when the tablet, pill or capsule is exposed to the gastrointestinal system is a highly crystalline fractureable water permeable material.

6. The oral drug delivery system of claim 5, wherein the highly crystalline fractureable water permeable material is selected from the group consisting of ethyl cellulose, cellulose acetate and polylactide/glycolide copolymer.

7. The oral drug delivery system of claim 1, wherein the sound generation means is a device capable of generating sound waves through electronic, hydraulic, or mechanical means.

8. The oral drug delivery system of claim 7, wherein the device is an electronic system capable of modulating the sound waves for the purpose of transmitting a serial number or a unique identifying signal.

9. The oral drug delivery system of claim 7, wherein the device generates an ultrasonic sound wave modulated in such a way as to transmit a serial number or a unique identifying signal.

10. The oral drug delivery system of any of claims 1-9, further comprising a sound sensor to be worn by a person so that when the person ingests the tablet, the sound sensor detects the sound waves produced when the tablet, pill or capsule is exposed to the gastrointestinal system.

11. The oral drug delivery system of claim 10, wherein the sound sensor is capable of demodulating the sound waves and recovering a transmitted serial number or unique identifying signal.

12. The oral drug delivery system of claim 10, wherein the sound sensor is an ultrasonic sound sensor.

13. The oral drug delivery system of claim 12, wherein the ultrasonic sound sensor is used to demodulate an ultrasonic sound wave, and recover a transmitted serial number or a unique identifying signal.

14. A method for oral drug compliance monitoring, comprising the steps of: (a) ingesting a tablet, pill or capsule comprising a material which produces sound waves when the tablet, pill or capsule is exposed to the gastrointestinal system of a person; and (b) detecting the sound waves produced when the tablet, pill or capsule is exposed to the gastrointestinal system to confirm that the person has ingested the tablet, pill or capsule.
